(19) United States
(12) Patent Application Publication
(10) Pub. No.: US 2023/0109708 A1
(43) Pub. Date: Apr. 13, 2023

(54) Cellular Uptake

(71) Applicant: Axcess (UK) Ltd, Liverpool (GB)

(72) Inventors: Roger R.C. New, London (GB); Glen Travers, London (GB)

(21) Appl. No.: 17/794,757

(22) PCT Filed: Jan. 22, 2021
(86) PCT No.: PCT/IB2021/050477

§ 371 Date: Jul. 22, 2022

(30) Foreign Application Priority Data
Jan. 23, 2020 (AU) 2020900183

(51) Int. Cl. A61K 31/185; A61K 47/14; A61K 47/18

(52) U.S. Cl. CPC A61K 31/185; A61K 47/14; A61K 47/183

(57) ABSTRACT

The present invention provides a bile acid or a pharmaceutically acceptable salt thereof, for use in a method of treatment of the human or animal body, which method comprises administering a therapeutic compound to the human or animal body, wherein: a. said bile acid is chenodeoxycholic acid or deoxycholic acid, and b. optionally, said bile salt is employed in conjunction with EDTA, c. in said method the bile acid or salt thereof, and EDTA, are used to enable or enhance intracellular uptake of the therapeutic compound.

### TECHNICAL FIELD

The present invention relates to methods of introducing material into the interior of biological cells, particularly intestinal cells. The methods are applicable to delivery into cells of therapeutic compounds, including macromolecules such as proteins, and can be used in the treatment of diseases involving the use of the therapeutic compounds to exert a therapeutic effect after entering the cell internal milieu.

### BACKGROUND ART

The following discussion of the background art is intended to facilitate an understanding of the present invention only. The discussion is not an acknowledgement or admission that any of the material referred to is or was part of the common general knowledge as at the priority date of the application.

Facilitating the uptake of therapeutic compounds into cells has been the focus of significant scientific interest due to the self-evident medicinal applications. A wide range of different approaches have been considered, but the challenges involved mean that there remains a considerable need for improved ways of achieving this aim.

Thus, biological cells have many specific mechanisms for regulating the uptake of materials into their interior, most often through the action of structure-selective receptors embedded in their surface membrane. In general, however, cellular architecture is designed so as to exclude hydrophilic molecules from the cell interior, since the phospholipid membrane enveloping all mammalian cells is essentially a hydrophobic barrier which prevents the passage of water-soluble molecules. This is particularly the case with macromolecules, and a large part of pharmaceutical research in recent years has been devoted to encouraging the entry of macromolecules into cells.

Intracellular uptake can obviously be useful for introducing therapeutic compounds which exert a beneficial effect inside the cell, as in e.g. gene therapy or enzyme replacement therapy. Gene therapy offers the promise of treating diseases via the production of therapeutic proteins within cells. Thus, for nucleic acid molecules that are used in gene therapy, the target sites are mostly inside the cells, in the cytoplasm or the nucleus, such that the molecules must traverse the plasma membrane to reach them. Most genetic molecules are both large and charged, though, making it difficult for them to traverse the plasma membrane on their own, so an appropriate gene delivery system is needed for efficient cellular uptake. Synthetic or nonviral gene delivery systems can circumvent some of the problems associated with viral vectors such as nonspecific inflammations and an unexpected immune response. Further, nonviral vectors have advantages in terms of simplicity of use and ease of large-scale production. However, the comparatively low efficiency is a major disadvantage of nonviral vectors, and efforts to address this are ongoing.

Among the various methodologies that have been developed so far with the aim of introducing material into the interior of biological cells, one involves the objective of reducing the polarity or hydrophilicity of the therapeutic compound, e.g. by complexing it with lipophilic moieties. Thus, enhancement of DNA uptake can be brought about by binding it electrostatically with cationic lipids such as lipofectin (Feigner P L (1991) Cationic liposome-mediated transfection with lipofectin™ reagent. Methods Mol Biol 7: 81-9). However, such methods suffer from a low level of efficiency, and, in the case of cationic lipids, high levels of cytotoxicity can be observed, depending on the cell type and reagent density, which make the technique undesirable for in vivo use. Issues also arise with stability and lack of reproducibility. For certain protein molecules, a similar effect can be achieved by chemically conjugating the protein with long-chain hydrocarbons, although such procedures may lead to enhanced immunogenicity of the proteins involved.

Another approach that has been considered, which is applicable particularly when the therapeutic compound is a macromolecule, is to encapsulate it inside a particulate carrier, e.g. a liposome, which can then be taken up readily by phagocytic cells. Such a technique was used with some success as a potential method of treatment for Gaucher's disease (a genetic deficiency of glucocerebrosidase), where this enzyme was incorporated into liposomes and delivered into cells lacking the ability to break down glucosyl ceramide (Belchetz P E, Crawley J C, Braidman I P, Gregoriadis G (1977) Treatment of Gaucher's disease with liposome-entrapped glucocerebroside: beta-glucosidase. Lancet. 16;2(8029):116-7). More recently, similar work has been carried out with beta-galactosidase (Umezawa F, Eto Y, Tokoro T, Ito F, Maekawa K. Enzyme replacement with liposomes containing beta-galactosidase from Charonia lumpas in murine globoid cell leukodystrophy (twitcher) Biochem Biophys Res Commun. 1985 Mar. 15;127(2):663-7) and transglutaminase (Aufenvenne K, Fernando Larcher, Ingrid Hausser, Blanca Duarte, Vinzenz Oji, Heike Nikolenko, Marcela Del Rio, Margitta Dathe and Heiko Traupe (2013) Topical Enzyme-Replacement Therapy Restores Transglutaminase 1 Activity and Corrects Architecture of Transglutaminase-1-Deficient Skin Grafts The American Journal of Human Genetics 93, 620-630, Oct. 3, 2013). This method is best suited to cells displaying significant phagocytic activity. For other cell types, specific targeting can be encouraged by incorporating polyethylene glycol-lipids and a target-specific antibody into the outer membrane. While this can assure binding of vesicles to the outer surface of the membrane, internalisation is not guaranteed.

Another approach, also applicable when the therapeutic compound is a protein, is to conjugate the protein of interest to a ligand which can interact specifically with a receptor molecule that will encourage the internalisation of the peptide. Examples of this approach are immunotoxins, where internalisation of the ricin A chain into a tumour is enhanced by linkage to an antibody molecule which targets to a tumour-specific antibody (M L Grossbard, J G Gribben, A S Freedman, J M Lambert, J Kinsella, S N Rabinowe, L Eliseo, J A Taylor, W A Blattler and CL Epstein (1993) Adjuvant Immunotoxin Therapy With Anti-B4-Blocked Ricin After Autologous Bone Marrow Transplantation for Patients With B-Cell Non-Hodgkin's Lymphoma Blood 81 2263-2271; and Antignani A and FitzGerald D. (2013) Immunotoxins: The Role of the Toxin Toxins 5 1486-1502). Abrin has been used in an immunotoxin for the same purpose (Gadadhar S, Karande A A (2013) Abrin Immunotoxin: Targeted Cytotoxicity and Intracellular Trafficking Pathway. PLoS ONE 8(3): e58304). However, this method is only applicable to agents with a very high potency, since the ratio of macromolecule to antibody is around 1:1.

As regards other possible approaches, some have started from the position that a number of pathways which allow small molecules (such as the degradation products of proteins that are formed by proteolysis) to cross enterocytes in a selective fashion have been described. Thus, approaches have been considered whereby therapeutic compounds such as macromolecules can be conjugated to such small molecules which may then be recognised by the relevant receptors, such that the conjugate can then be pulled across the membrane in its entirety. These pathways, making use of specific receptors on the cell surface, include vitamin uptake routes, such as for vitamin B12 or biotin (making use of the dipeptidyl receptor), and routes involving receptors for conjugated bile salts.

In the case of the dipeptidyl receptor, the receptor density can be affected by environmental conditions, and acts at a relatively low speed, since overall daily requirements for these vitamins are low, in terms of quantity, so the amount of material delivered is limited.

For the bile salt receptor, in contrast, tens of grams of material can be internalised every day. Thus, the potential to be exploited as a delivery mechanism is great, and approaches have been described in which compounds (to be delivered) have been attached to bile salts, and enhanced uptake of the resulting conjugates into cells shown. Generally, the molecules attached to the bile acids have been small, low-molecular weight entities, although a limited amount of success has been claimed also for certain peptides (see e.g. U.S. Pat. No. 7,153,930). In this regard, the general thinking has been that the bile salt needs to be in monomeric form (i.e. not associated with further bile salt molecules) in order to bind to the relevant receptors, and that the therapeutic compound must be covalently bonded to the bile salt. Indeed, given that the binding between the bile salt and its receptor takes place via non-covalent interactions, a single bile salt molecule may generally be expected to be too small to also interact with the therapeutic compound via a non-covalent interaction.

### SUMMARY OF INVENTION

The present invention concerns a new approach to facilitating the intracellular uptake of compounds. The invention is based on the surprising finding that certain bile acids and/or pharmaceutically acceptable salts thereof (referred to herein also as “the bile acid(s)/salt(s)”) can interact with receptors on the cell surface (interestingly this effect is seen only for two particular unconjugated bile acids/salts, namely chenodeoxcholic acid and deoxycholic acid and their salts—it is not seen with conjugated bile acids/salts, or indeed with the other unconjugated bile acids/salts). In this regard, it can be envisaged that bile salts, in particular when in the form of micelles, can interact with more than one receptor at a time, and that the resultant receptor aggregation on the membrane surface triggers membrane invagination and internalisation (e.g. of, inter alia, the receptors and micelle), i.e. intracellular uptake of material from outside the cell. The present invention concerns the exploitation of this newly found pathway in order to facilitate the uptake of material into cells, in particular through a process in which vesiculation is stimulated.

Preferably, in the methods of the present invention described herein, bile acid or salt receptor-mediated internalisation via clathrin-coated pits is used to enable or enhance intracellular uptake of the therapeutic compound. Thus, the mechanisms whereby bile acid/salts interact with cell surface receptors, can trigger internalisation processes which may be utilised to enable or enhance intracellular uptake of a therapeutic compound, which can even be a macromolecule. In this regard, in one aspect of the invention, intracellular uptake of the therapeutic compound may be achieved by arranging for the therapeutic compound to be in close proximity to the cell surface at the same time as the bile acid/salt. Thus, in a preferred embodiment of the method of the invention, bile acid or salt receptor-mediated internalisation via clathrin-coated pits is used to enable or enhance intracellular uptake of one or more therapeutic compounds located in the vicinity of the cell (but not conjugated to the bile acid/salt). The process is concentration dependent, and although the bile acid or salt is generally used at concentrations that are relatively high, efficacy can be achieved at concentrations which are not toxic to the cells. Fluorescence tests have shown that the bile acid or salt enables or enhances uptake of even large (macromolecular) therapeutic compounds, which are then visible in vacuoles within cells. Tests have also shown that including an inhibitor of clathrin has the effect of blocking the intracellular absorption enhancing effect of the bile acid or salt, indicating that the mechanism of action of the bile acid or salt is clathrin-mediated endocytosis.

It has also been found that EDTA can have an additional enhancing effect on uptake of protein stimulated by chenodeoxycholate. Unexpectedly, while EDTA alone, at non-toxic concentrations, has no effect on uptake, it is able to enhance the stimulation seen with chenodeoxycholate, and in some cases, uptake is seen with these two agents in combination even when no uptake is observed with those agents employed separately at the same concentrations. Thus, it appears that EDTA is able to synergise with chenodeoxycholate, to enhance uptake of protein into intestinal cells. It seems unlikely that this phenomenon is brought about simply by a chelating activity, since other agents which, like EDTA, are known to exhibit chelating activity (eg DTPA, EGTA, ortho-phenanthroline), do not show the same effect as EDTA. It is a further feature of this invention that a formulation comprising a combination of chenodeoxycholate and EDTA can be used to enhance protein uptake by intestinal cells, and this combination can provide an even more effective means of achieving uptake by cells than chenodeoxycholate alone. In this way, uptake by and passage across the intestinal cell barrier can be affected, resulting in introduction of macromolecules into the body, as a result of administration via the oral route. The concentration of the EDTA which is achieved at the surface of the cells into which intracellular uptake is to be enabled or enhanced, can range from 0.1 mg/ml to 10 mg/ml, and preferably from 0.2 mg/ml to 10 mg/ml.

Prior to the present invention, it is believed that it was not known that bile acids/salts could assist intracellular uptake of macromolecular therapeutic compounds. Rather, it was generally assumed that uptake would not work unless the bile salt was in the form of a monomer covalently bonded to the therapeutic compound. It is also believed that it was not known that receptors could bind to and be activated by either of the two the specific bile salts chenodeoxycholate and deoxycholate. Thus, the understanding in the scientific literature has been that such unconjugated bile salts are taken up from the lumen of the gut into the body principally by passive diffusion across the cell membrane of enterocytes, see e.g. Trauner et al (Physiol Rev, Vol 83, April 2003), Stamp et al (An Overview of Bile-Acid Synthesis, Chemistry and Function, Issues in Toxicology, Bile Acids: Toxicology and Bioactivity, Royal Society of Chemistry, 2008), Michael W King, PhD (Bile Acid Synthesis and Utilization, 1996-2014 © themedicalbiochemistrypage.org) and Dawson et al (J Lipid Res. 2009 December; 50(12): 2340-2357), Dawson & Karpen (Journal of Lipid Research, Volume 56, 2015, pages 1085-1099— see in particular FIG. 1 thereof which indicates that there appears to be no known take up of chenodeoxycholate in the unconjugated form by receptors in the small intestine), Ferrebee et al (Acta Pharmaceutica Sinica B 2015; 5(2):129-134), and Ninomiya et al (Biochimica et Biophysica Acta 1634 (2003) 116-125). In contrast, the conjugated tauro- or glycol- bile acids and salts are said to require an active transport mechanism—e.g. conjugated bile acid/salt uptake has been described previously as operating via specific receptors—and it has been suggested that these receptors may be able to recognise unconjugated bile salts but that their affinity is greatest for conjugated bile salts. Thus, it is believed that there is no report in the literature of receptors which bind to and are activated by two specific unconjugated bile acids/salts (chenodeoxycholate and deoxycholate) in the circumstances described herein, while also not binding to and/or being activated by either (i) conjugated bile acids/salts, or (ii) the other unconjugated bile acids/salts.

In this regard, as background relating to the bile acids/salts for use in the present invention, bile acids as a general class are among a wide range of different types of compound which have been discussed previously for possible use as agents that may help facilitate the absorption of therapeutic compounds across cellular barriers. By way of illustration, types of compound that have been described for this purpose include chelating agents such as EDTA or EGTA; non-ionic surfactants such as polyoxyethylene ethers, p-t-octyl phenol polyoxyethylenes, nonylphenoxy- polyoxyethylenes, and polyoxyethylene sorbitan esters; anionic agents such as cholesterol derivatives (including bile acids); cationic agents such as acylcarnitines, acylcholines, lauroylcholine, cetyl pyridinium chlorides and cationic phospholipids; plus other agents such as α-galactosidase, β-mannanase, sodium caprate, sodium salicylate, n-dodecyl-β-D-maltopyranoside, N,N,N-trimethyl chitosan chloride (TMC), cyclodextrin and NO donating compounds.

In terms of the use of bile salts as absorption enhancers, a recent review is set out in Moghimipour et al (Absorption-Enhancing Effects of Bile Salts, Molecules 2015, 20, 14451-14473). As is evident from Table 1 in that paper, attention has focused on conjugated bile acids, i.e. bile acids with a glyco- or tauro- group at the C-24 position (this may in part be due to the fact that conjugated salts have been reported to have superior emulsification properties as compared to unconjugated bile salts). For instance, in relation to oral drug delivery, some mixed results for sodium taurodeoxycholate have been described along with some positive results for sodium glycocholate (see pages 14457 and 14458 of Moghimipour et al).

As regards possible mechanisms of action that have been suggested for how bile acids may enhance absorption, these include causing damage to/the opening of tight junctions between epithelial cells so as to facilitate paracellular transport (e.g. via the binding of Ca2+ in the intercellular region, the disruption of hemidesmosomes, an interaction with filamentous actin, and/or the formation of reverse micelles), and protease inhibition. Some papers concerned with pulmonary and buccal routes of administration have noted that while bile salts can enhance paracellular transport in a concentration dependent manner, there have been cases where particularly high concentrations of certain conjugated bile salts have also encouraged the uptake of a proportion of the material into the cells (Nicolazzo et al, Journal of Controlled Release 105 (2005) 1-15; Hoogstraate et al, Journal of Controlled Release 40 (1996) 211-221; Hussain et al, Journal of Controlled Release 94 (2004) 15-24; and Dodla et al, Asian J Pharm Clin Res, Vol 6, Issue 3, 2013, 39-47). However, this is suggested to be due to disruption/perturbation of the cell membrane, e.g. by lipid extraction, which is not a desirable approach for delivering therapeutic compounds into cells. Thus, even notwithstanding the potential deleterious effect on the membrane (e.g. by perturbing vital cell structures and/or functions) and the fact that significant proportions of the compounds follow an intercellular route in these instances, breaching the membrane in this manner may pave the way for the inward penetration of toxic or otherwise undesirable material that may be located in the vicinity of the disrupted area. Moreover, the high concentrations of the conjugated bile acids that need to be employed before such effects are seen to a relevant extent are in any event generally too high from a toxicity point of view.

As noted above, the present invention is based on the surprising finding that two unconjugated bile acids/salts, namely chenodeoxcholic acid and deoxycholic acid and their salts, are able to facilitate the uptake of material into cells by interacting with receptors on the cell membrane (while other bile salts, including the more popular conjugated bile salts, are not able to do this). In accordance with the present invention, the bile acids/salts may thus be used to induce the uptake of therapeutic compounds such as macromolecules, to assist in them reaching internal parts of the cell such as the cytoplasm or nucleus. Further, the effect is concentration dependent, but while the bile acid/salt is generally used at concentrations that are relatively high, efficacy has been achieved at concentrations which are not toxic to the cells. Testing has shown that the bile acid/salt enables or enhances the uptake of even large (macromolecular) therapeutic compounds, which are then visible in vacuoles within cells. It has also been shown that including an inhibitor of clathrin has the effect of blocking the intracellular absorption enhancing effect of the bile acid/salt, indicating that the mechanism of action is clathrin-mediated endocytosis.

Thus, the present invention provides a bile acid or a pharmaceutically acceptable salt thereof, for use in a method of treatment of a human or animal body, which method comprises the step of: administering a therapeutic compound to the human or animal body, together with a bile acid, wherein said bile acid is chenodeoxycholic acid or deoxycholic acid.

Preferably, the bile acid or salt thereof is used to enable or enhance intracellular uptake of the therapeutic compound. Optionally, said bile salt is employed in conjunction with EDTA.

The present invention also provides a method of treatment of the human or animal body, which method comprises the step of: administering a therapeutic compound to the human or animal body together with together with a bile acid, wherein said bile acid is chenodeoxycholic acid or deoxycholic acid, wherein the bile acid or a pharmaceutically acceptable salt thereof is present in an amount that is sufficient to enable or enhance intracellular uptake of the therapeutic compound. Optionally, said bile salt is employed in conjunction with EDTA.

The present invention also provides for the use of a therapeutic compound and a bile acid or salt thereof in the manufacture of a medicament for use in a method of treatment of a human or animal body in need of the therapeutic compound. Preferably, the bile acid or salt thereof is present in an amount to enable or enhance intracellular uptake of the therapeutic compound. Optionally, said bile salt is employed in conjunction with EDTA

The present invention also provides a pharmaceutical or therapeutic composition comprising (a) a bile acid or a pharmaceutically acceptable salt thereof, wherein said bile acid is chenodeoxycholic acid or deoxycholic acid; and (b) a therapeutic compound. Preferably the composition also includes one or more further compounds selected from fusogenic lipids, cell penetrating peptides, lysosomotropic agents, membrane-disruptive peptides, membrane-disruptive polymers, photochemical internalisation agents, and agents that alter intracellular vesicle transport, and, optionally, said bile salt is employed in conjunction with EDTA, to this effect.

Desirably, in any formulation of the invention the bile acid or salt and therapeutic compound are encapsulated by a coating which (a) restricts dissolution of the bile acid or salt and therapeutic compound under aqueous conditions at a pH of 7.4, but ceases to restrict dissolution of the bile acid or salt and therapeutic compound at a pH which is lower than 7.4, and/or (b) contains one or more moieties capable of binding to a target cell population within the body.

As indicated above, the methods of treatment of the present invention described herein involve administrating a therapeutic compound to the human or animal body. In this regard, as a general matter, it is of course intended that said therapeutic compound then provides a therapeutic effect which underlies the method of treatment.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 shows photomicrographs of Caco-2 cells after incubation with medium containing (i) FITC-insulin alone (concentration 100 ug/ml), (ii) FITC-insulin with sodium chenodeoxycholate (1 mg/ml) and (iii) FITC-insulin with sodium chenodeoxycholate (2 mg/ml). These show that very little uptake occurs in cells treated with insulin alone, but that uptake is enhanced by the presence of chenodeoxycholate in a concentration-dependent manner.

FIG. 2 shows the relative uptake of fluorescent albumin into Caco-2 cells, at different time points, in (i) the absence of chenodeoxycholate and propyl gallate, (ii) the presence of chenodeoxycholate but the absence of propyl gallate, and (iii) the presence of low, medium and high concentrations of (both) chenodeoxycholate and propyl gallate.

FIG. 3 shows the different effects of a range of bile salts on the uptake of fluorescent albumin into Caco-2 cells.

FIG. 4. shows the results for uptake of FITC-BSA which demonstrates that chlorpromazine (at both high and low concentrations) inhibits uptake stimulated by the bile salt at all concentrations for cells adhering to the wells of a microplate.

FIG. 5 shows the results for uptake of FITC-BSA which demonstrates that chlorpromazine (at both high and low concentrations) inhibits uptake stimulated by the bile salt at all concentrations for cells in suspension.

FIG. 6. shows that when chenodeoxycholate and EDTA are combined, a very significant enhancement of uptake occurs.

FIG. 7 shows that chenodeoxycholate enhances the uptake of hGH into cells in a dose-dependent manner similar to that seen for uptake of other proteins such as insulin, BSA and casein.

FIG. 8 shows that, in a similar manner to that seen for Caco-2 cells, uptake of protein into IEC6 cells is enhanced by the presence of chenodeoxycholate, confirming that this is a phenomenon common to intestinal cells in general.

FIG. 9 shows that, in IEC6 cells enhancement of uptake of BSA by chenodeoxycholate is augmented by EDTA in a dose-dependent fashion.

### DESCRIPTION OF EMBODIMENTS

For convenience, the following sections generally outline the various meanings of the terms used herein. Following this discussion, general aspects regarding compositions, use of medicaments and methods of the invention are discussed, followed by specific examples demonstrating the properties of various embodiments of the invention and how they can be employed.

### Definitions

The meaning of certain terms and phrases used in the specification, examples, and appended claims, are provided below. If there is an apparent discrepancy between the usage of a term in the art and its definition provided herein, the definition provided within the specification shall prevail.

Those skilled in the art will appreciate that the invention described herein is susceptible to variations and modifications other than those specifically described. The invention includes all such variations and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness. None of the cited material or the information contained in that material should, however be understood to be common general knowledge.

Manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

Other than in the operating examples, or where otherwise indicated, all numbers expressing quantities of ingredients or reaction conditions used herein should be understood as modified in all instances by the term “about.” The term “about” when used in connection with percentages can mean±1%.

The invention described herein may include one or more range of values (e.g. size, concentration etc.). A range of values will be understood to include all values within the range, including the values defining the range, and values adjacent to the range which lead to the same or substantially the same outcome as the values immediately adjacent to that value which defines the boundary to the range. For example, a person skilled in the field will understand that a 10% variation in upper or lower limits of a range can be totally appropriate and is encompassed by the invention. More particularly, the variation in upper or lower limits of a range will be 5% or as is commonly recognised in the art, whichever is greater.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

Throughout this specification, unless the context requires otherwise, the word “comprise” or variations such as “comprises” or “comprising”, will be understood to imply the inclusion of a stated integer or group of integers but not the exclusion of any other integer or group of integers.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, e.g. in the absence of an agent, for example a decrease by at least about 20%, or at least about 30%, or at least about 40%, or at least about 50%), or at least about 60%>, or at least about 70%, or at least about 80%.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, e.g. in in the absence of an agent, for example an increase of at least about 20%, or at least about 30%, or at least about 40%, or at least about 50%), or at least about 60%, or at least about 70%, or at least about 80%, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration. In certain embodiments, the compound is administered by parenterally administration, or other method allowing delivery to a target site.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Features of the invention will now be discussed with reference to the following non-limiting description and examples.

### EMBODIMENTS

Thus, the present invention provides a bile acid or a pharmaceutically acceptable salt thereof, for use in a method of treatment of a human or animal body, which method comprises the step of: administering a therapeutic compound to the human or animal body, together with a bile acid, wherein said bile acid is chenodeoxycholic acid or deoxycholic acid.

In this regard, the bile acid is preferably chenodeoxycholic acid. Also, it is preferable to use the bile acid in salt form. Thus, preferably said bile acid or a pharmaceutically acceptable salt thereof is a salt of chenodeoxycholic acid or deoxycholic acid, more preferably a salt of chenodeoxycholic acid.

The pharmaceutically acceptable salts of the bile acids are typically salts with a pharmaceutically acceptable base. Pharmaceutically acceptable bases include alkali metals (e.g. sodium or potassium), alkali earth metals (e.g. calcium or magnesium), hydroxides, and organic bases such as alkyl amines, aralkyl amines and heterocyclic amines. The alkali metals are preferred, particularly sodium. Thus, most preferably said bile acid or a pharmaceutically acceptable salt thereof is sodium chenodeoxycholate.

It is possible for more than one of the above bile acids and/or salts to be present, e.g. two, three or more may be present, preferably two or three, and more preferably two. Typically, though, only one is present.

The bile acid or salt thereof for use in the present invention is preferably comprised in a pharmaceutical composition.

The therapeutic compound for use in accordance with the present invention may be any compound that has a therapeutic effect inside a human or animal body, including inside one or more cells in the human or animal body. In this regard, and generally herein, references to a (or the) therapeutic compound for use in accordance with the invention are intended to encompass also the possibility of using more than one therapeutic compound, such as 2, 3, or more therapeutic compounds. Typically, though, references to a (or the) therapeutic compound preferably mean just one therapeutic compound.

As noted above, the effect underlying the present invention is sufficiently robust that it can even enable the intracellular uptake of large macromolecules. Given that enabling and/or enhancing the uptake of such physically large molecules into cells can be difficult, the effectiveness of the present invention is particularly useful in this context. Thus, preferably the therapeutic compound for use in accordance with the present invention is a macromolecule. In this regard, the therapeutic compound preferably has a molecular weight of around 1000 Da or more, such as e.g. 2000 Da or more, or 3000 Da or more.

The therapeutic compound is preferably, although not essentially, a peptide, more preferably a polypeptide, and more preferably still is a protein. Examples of suitable therapeutic compounds include, without limitation, insulin; calcitonin; human serum albumin; growth hormone; growth hormone releasing factors; galanin; parathyroid hormone; peptide YY; oxyntomodulin; blood clotting proteins such as kinogen, prothombin, fibrinogen, Factor VII, Factor VIII of Factor IX; erythropoietin and EPO mimetics; colony stimulating factors including GCSF and GMCSF; platelet-derived growth factors; epidermal growth factors; fibroblast growth factors; transforming growth factors; GLP-1, GLP-2; GLP-1 analogues and fusion proteins, GIP, glucagon; exendin; leptin; GAG; cytokines; insulin-like growth factors; bone- and cartilage-inducing factors; neurotrophic factors; interleukins including IL-1, IL-2, IL-3, IL-4, IL-5, IL-6, IL-7, IL-8, IL-9, IL-10, IL-11, IL-12; interferons including interferon gamma, interferon-1a, interferon alphas; TNF alpha; TNF beta; TGF-beta; cholera toxin A and B fragments; E. coli enterotoxin A and B fragments; secretin; enzymes including histone deacetylase, superoxide dismutase, catalase, adenosine deaminase, thymidine kinase, cytosine deaminase, proteases, lipases, carbohydrases, nucleotidases, polymerases, kinases and phosphatases; transport or binding proteins especially those which bind and/or transport a vitamin, metal ion, amino acid or lipid or lipoprotein such as cholesterol ester transfer protein, phospholipid transfer protein, HDL binding protein; connective tissue proteins such as a collagen, elastin or fibronectin; a muscle protein such as actin, myosin, dystrophin, or mini-dystrophin; a neuronal, liver, cardiac, or adipocyte protein; a cytotoxic protein; a cytochrome; a protein which is able to cause replication, growth or differentiation of cells; a signalling molecule such as an intra-cellular signalling protein or an extracellular signalling protein (eg hormone); trophic factors such as BDNF, CNTFSNGF, IGF, GMF, aFGF, bFGF, VEGF, NT3, T3 and HARP; apolipoproteins; antibody molecules, antibody fragments, single-domain antibodies; receptors in soluble form such as T-cell receptors and receptors for cytokines, interferons or chemokines; proteins or peptides containing antigenic epitopes and fragments; and albumin fusion proteins, derivatives, conjugates and sequence variants of any of the above. These and other proteins may be derived from human, plant, animal, bacterial or fungal sources, and extracted either from natural sources, prepared as recombinants by fermentation or chemically synthesised. In a preferred aspect, the therapeutic compound is a peptide selected from insulin, calcitonin, growth hormone, growth hormone releasing factors, galanin, parathyroid hormone, peptide YY, oxyntomodulin, erythropoietin, colony stimulating factors, platelet-derived growth factors, epidermal growth factors, fibroblast growth factors, transforming growth factors, GLP-1, GLP-2, GIP, glucagon, exendin, leptin, neurotrophic factors, insulin-like growth factors, cartilage-inducing factors, IL-1, IL-2, IL-3, IL-4, IL-5, IL-6, IL-7, IL-8, IL-9, IL-10, IL-11, IL-12, interferon-gamma, interferon-1a, interferon alphas, and conjugates of any of the above, fusion proteins including any of the above, and any of the above in combination.

The therapeutic compound for use in the present invention is preferably comprised in a pharmaceutical composition.

It is possible for the bile acid/salt and the therapeutic compound (and/or, if present, any additional agents for use in the method) to be administered separately. Preferably, though, said method of the present invention comprises administering a composition comprising (both) the bile acid/salt and the therapeutic compound. Further, if one or more additional agents are being used in combination with the bile acid/salt in the method, said composition preferably also comprises one or more of said one or more additional agents, and typically all of said one or more additional agents.

In the method of the present invention, the upper limit for the concentration of the bile acid/salt that is desired at the surface of the cells (into which uptake of the therapeutic compound is to be enabled or enhanced) may be determined by the level at which the bile acid/salt is not toxic, in vivo, to the (target) cell type and/or surrounding cells. Thus, in the method of the present invention, the concentration of the bile acid/salt which is achieved at the surface of the cells into which intracellular uptake is to be enabled or enhanced should be below the level at which it would be toxic to the (target) cell type and/or surrounding cells.

In the method of the present invention, the lower limit for the concentration of the bile acid/salt that is desired at the surface of the cells (into which uptake of the therapeutic compound is to be enabled or enhanced) may preferably correspond to the critical micelle concentration (CMC), i.e. the concentration above which the bile acid/salt forms micelles. The exact CMC can vary depending on, inter alia, temperature, pressure, and the presence and concentration of other agents (particularly surface active substances and electrolytes) and can be determined by experiment. Thus, in the method of the present invention, the concentration of the bile acid/salt which is achieved at the surface of the cells into which intracellular uptake is to be enabled or enhanced is preferably above the CMC of the bile acid/salt (in that particular environment).

Generally, in the method of the present invention, the concentration of the bile acid/salt which is achieved at the surface of the cells (into which uptake of the therapeutic compound is to be enabled or enhanced) preferably ranges from 0.1 mg/ml to 500 mg/ml, preferably from 0.5 mg/ml to 100 mg/ml, more preferably from 1 mg/ml to 50 mg/ml and yet more preferably from 2 mg/ml to 40 mg/ml, such as 5 mg/ml to 20 mg/ml.

The amount of the bile acid/salt to include in a pharmaceutical composition for administration to the patient is preferably chosen so as to attain such concentrations in the vicinity of the cells into which uptake is desired. This may vary depending on e.g. the mode of administration and the nature and size of the target site. The skilled person will be able to adapt the concentration accordingly.

In one aspect, the amount of bile acid/salt may be from 0.1 mg to 500 mg, preferably from 0.5 mg to 100 mg, more preferably from 1 mg to 50 mg and yet more preferably from 2 mg to 40 mg, such as 5 mg to 20 mg, per cubic centimetre of target cells (e.g. tumour cells). These amounts are particularly useful if the bile acid or salt and therapeutic compound are comprised in one composition, which is for administration to the blood stream, e.g. intravenously, in a form which will release the bile acid or salt and the therapeutic compound at a specific location within the body, e.g. in the acidic environment of tumour cells.

In another aspect, the amount of bile acid/salt in the composition may be at least 0.1 mg, preferably at least 1 mg, more preferably at least 10 mg, yet more preferably at least 20 or at least 50 mg. The amount of bile acid/salt in the composition may be up to 1 g, preferably up to 500 mg, more preferably up to 200 mg and yet more preferably up to 100 mg. A typical amount is 50 to 100 mg, such as e.g. 60 to 80 mg, or around 70 mg. These amounts are particularly useful if the bile acid/salt and therapeutic compound are comprised in an oral composition for effecting uptake of the therapeutic compound and the bile acid/salt into the intestinal cells (e.g. by having an enteric coating to avoid the contents being released in the stomach), since when the contents of the composition are released in the intestine this will typically result in them becoming dispersed primarily in a localised aqueous volume of a few ml, e.g. up to around 5 ml.

The amount of the therapeutic compound to use in accordance with the present invention may depend on the underlying disease or condition, the nature and size of the target cell population, the type and severity of the disease, the therapeutic compound, the age, weight and condition of the patient, and the mode plus frequency of administration. The skilled medical practitioner will be able to select appropriate amounts, but typical dosage levels for the therapeutic compound are 0.01 to 100 mg/kg, such as 0.1 to 10 mg/kg.

The ratio of the bile acid/salt:therapeutic compound to be administered in combination in accordance with the present invention is preferably 100:1 to 1:1 weight-to-weight, more preferably 70:1 to 3:2, yet more preferably 50:1 to 2:1, more preferably still 30:1 to 4:1, and most preferably 20:1 to 5:1, such as e.g. 15:1 to 10:1.

As mentioned above, the methods of the present invention involve the use of the bile acid/salt to enable or enhance intracellular uptake of the therapeutic compound, and preferably, bile acid or salt receptor-mediated internalisation via clathrin-coated pits is used to enable or enhance said intracellular uptake. In this regard, in one aspect, intracellular uptake may be achieved by arranging for the therapeutic compound to be in sufficiently close proximity to the cell surface at the same time as the bile acid/salt. In a second (not mutually exclusive) aspect, though, internalisation may be brought about by associating the therapeutic compound directly with the bile acid/salt (e.g. in the form of micelles) via a non-covalent interaction. The following embodiments are relevant for both aspects, although in some instances it will be apparent that a given embodiment is particularly relevant for the second of these two aspects of the invention.

Thus, in a preferred embodiment of the method of the present invention, the bile acid/salt is used in combination with one or more additional agents.

In one embodiment, said one or more additional agents comprise one or more agents which:
a) stabilise a micellar form of the bile acid or salt thereof (against disassociation); and/or
b) enable or enhance the formation of a micellar form of the bile acid or salt thereof.

Suitable agents for use in this regard include agents which are, or which comprise, hydrophobic entities. The hydrophobic entities can be incorporated within the micelle thus creating a situation where breakdown of the micelle is energetically unfavourable in an aqueous environment, since it would lead to exposure of the hydrophobic entities to water. In one embodiment, the therapeutic compound itself may serve to stabilise and/or enable or enhance the formation of the micellar form to some extent at least, e.g. if the therapeutic compound contains a hydrophobic part (such as a lipid tail composed of a long-chain hydrocarbon). In this embodiment it may be unnecessary to include an additional agent.

In any event, though, it is generally advantageous to include one or more additional (separate) agents which stabilise the micellar form and/or enable or enhance its formation, as this can help facilitate the internalisation process. Agents which may be used in this regard generally include any agent having a hydrophobic moiety such as a long (e.g. C4 or more, C5 or more, or C6 or more, and up to e.g. C30, C20 or C12) hydrocarbyl chain. Examples include fatty acids or steroids such as cholesterol (i.e. the results of breakdown of lipidic structures in food), and hydrophobic antioxidants such as aromatic alcohols including propyl gallate and butylated hydroxyl anisole. Propyl gallate is particularly preferred.

In this regard, in embodiments of the invention where the bile acid/salt and therapeutic compound are comprised in a single composition, and said composition is solid, the amount of said one or more additional (separate) agents which stabilise the micellar form and/or enable or enhance its formation (preferably propyl gallate), if present, is preferably at least 1 mg, such as at least 10 mg or at least 20 mg. The amount may be up to 150 mg such as up to 100 mg or up to 50 mg. A typical amount is 20 to 50 mg, such as 30 to 40 mg.

As regards the size of the micelles, this is not particularly limited, but the micelle will have an aggregation size of at least 2 molecules, preferably at least 3, more preferably at least 4. The aggregation size is generally 30 molecules or less, preferably 20 or less, more preferably 10 or less, and typically 8 or less. Average aggregation sizes of around 5 to 7 molecules (e.g. around 6 molecules) are particularly preferred.

In some embodiments said one or more additional agents for use in the invention may include a pH adjuster such as carbonate or bicarbonate salt. This can help improve the solubility of the bile acid/salt thereof, e.g. by elevating the pH to 7.5 to 9.

In accordance with the present invention, the bile acid/salt and the therapeutic compound are used in combination. There is no particular limitation on how the bile acid/salt and the therapeutic compound are administered, provided that they are administered so as to give rise to the situation where they are both present at the same time and at a sufficiently high concentration in the vicinity of the cells into which uptake is desired. The bile acid/salt and the therapeutic compound may be administered separately, simultaneously, or as part of a single composition. As noted above, preferably the bile acid/salt and therapeutic compound are combined in a single composition prior to administration. In this regard, the bile acid/salt may simply be admixed with the therapeutic compound.

In the method of the present invention, intracellular uptake is believed to take place by clathrin mediated endocytosis—in other words, by the formation of clathrin-coated pits. Thus, as reported in the Examples below, it has been found that uptake is inhibited by chlorpromazine (an inhibitor of clathrin-coated pit formation) but not by nystatin (caveoli) or amiloride (mcaropinocytosis). The pit formation is believed to be a receptor-mediated process in which the bile acid/salt binds specifically to a cell-surface receptor.

Thus, the present invention is generally applicable to any method of treatment that requires the intracellular delivery of a therapeutic compound. The finding of this new effect of the bile acids/salts thus opens up new therapies, e.g. for treatments in which previously it was not viable practically and/or economically to achieve the effective intracellular delivery of a given therapeutic compound to a target cell population in patients.

As regards the mode of administration to be employed in the method of the present invention, this should be selected such that the therapeutic compound and bile acid/salt are delivered to the target cell population before contacting any other cells. One way of effecting this is just to administer the bile acid/salt and the therapeutic compound directly to the relevant location. Thus, if the location was the skin, this could be achieved with a topical formulation, or if the location was the nasal cavity or lungs this could be done using a spray or inhaler. Locations within the body may be reached by injection or keyhole-type delivery/release.

However, it is not necessary for the target cell population to be present at the site of administration. Thus, the bile acid/salt and the therapeutic compound may be formulated in one or more compositions which delay their contact with surrounding cells until the composition(s) reaches the target cell population. This enables a high concentration of the bile acid/salt and the therapeutic compound to be achieved at the surface of the target cells, while also avoiding elevated concentrations elsewhere in the body.

In this regard the bile acid/salt and therapeutic compound may be administered e.g. parenterally, such as by intravenous injection. An instance where this mode of administration is preferred, is for embodiments of the invention wherein the bile acid/salt and therapeutic compound are encapsulated by a coating which (a) restricts dissolution of the bile acid/salt and therapeutic compound under aqueous conditions at a pH of 7.4 (pH 7.4 is physiological pH), but ceases to restrict dissolution of the bile acid or salt and therapeutic compound at a pH which is lower than 7.4, and/or (b) contains one or more moieties capable of binding to a target cell population within the body. The coating may be designed e.g. so as to become permeable or break down in a particular environment, e.g. within a certain pH range. This approach may be used e.g. in the treatment of cancer, by encapsulating the therapeutic compound and bile acid/salt within a coating that will release its cargo in the acidic microenvironment of tumour tissue, e.g. at a pH of lower than 7.4. For instance, the coating could be designed to release the therapeutic compound and the bile acid or salt at a pH of around 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, or 7.3, or indeed anywhere within the range of 5.0 to 7.3 or any sub-range based on the intervening figures given above. The precise pH will vary according to the tumour. It may be appropriate to test the pH in the environment of the tumour cells before treatment, to check what formulation is most desirable. Possible formulations that could be used in this regard include micronised pellets containing a coating which (a) restricts dissolution of the pellet, but which can be formulated so as to dissolve in such acidic microenvironments, and/or (b) contains one or more molecules or moieties capable of binding to a receptor present at the target cell population, e.g. cancer cells (in this regard it can also be advantageous if, when the molecules reach the target site and the relevant moieties bind to the intended receptors, the release of the composition from within the coating may be triggered/encouraged). In one aspect, cancer cells may be targeted by using the bile acid/salt and the therapeutic compound in combination with DOPE and PEG. In this case, the acid-sensitive PEG breaks down in the acidic tumour environment to expose the bile acid/salt and the therapeutic compound to the target cells.

The bile acid/salt and therapeutic compound may also be administered orally. For instance if the intended site of intracellular uptake of the therapeutic compound is the cells of the small intestine, then a safe passage through the stomach can be achieved by encapsulating them inside an enteric-coated capsule, tablet or other device which resists dissolution at the low pH found in the stomach, but disintegrates at higher pH to release the compound into the small intestine, e.g. the duodenum, jejunum or ileum. This protects against dissolution of the bile acid/salt and the therapeutic compound (to concentrations that are too low for the intracellular uptake to occur effectively) and/or against breakdown of one or more of the components (particularly the therapeutic compound) in the stomach. Pharmaceutical compositions for use in accordance with the present invention in this regard preferably have an enteric coating which becomes permeable at a pH of from 3 to 7, more preferably 4 to 6.5, and most preferably 5 to 6. Suitable enteric coatings are known in the art.

The fate of a therapeutic compound after intracellular uptake may depend on factors including of course the nature of the compound itself, any other compounds that have also been taken up into the cell with it, and the nature of the cell into which uptake has occurred. For instance, some cells have an intrinsic function of transporting material ingested in vesicles across the cell for subsequent departure (e.g. barrier cells such as an endothelial cells in the blood brain barrier). In some contexts, this may of course serve a useful function. However, in situations where the therapeutic compound is intended to have a therapeutic effect within that cell, this illustrates the sort of situation where it can be particularly advantageous to take an additional measure in order to help facilitate that effect.

Thus, as mentioned above, in the method of the present invention, the bile acid or salt thereof may preferably by used in combination with one or more additional agents. In this regard, in a preferred embodiment, said one or more additional agents comprise one or more agents which act to increase the likelihood of the therapeutic compound successfully exerting its therapeutic effect in the cell (into which uptake occurs) by one or more of the following mechanisms:
a) inhibiting the subsequent departure of the therapeutic compound from the cell after it has been taken up,
b) protecting the therapeutic compound against the acidic pH of endosomes/lysosomes,
c) protecting the therapeutic compound against one or more digestive enzymes of lysosomes,
d) promoting penetration of the endosomal barrier (facilitating endosomal escape), e) increasing the stability of the therapeutic compound in the cytosol,
f) promoting penetration of the nuclear membrane (if the intended site of action is the nucleus, e.g. for plasmid DNA delivery), and
g) altering intracellular vesicle transport.

For instance, said one or more additional agents may comprise one or more agents selected from the following:
a) Fusogenic lipids, e.g. cationic liposomes and/or neutral helper lipids. Cationic liposomes may advantageously be used when the therapeutic compound is a nucleic acid, to form a complex with the nucleic acid, which can encourage gene transfection. As regards neutral helper lipids, agents such as DOPE (dioleoylphosphatidylethanol-amine) can advantageously be used. DOPE can mediate fusion between the liposomes and the endosomal membrane following endocytosis, and so may be used e.g. to enhance the expression of a complexed gene. When the neutral helper lipid is DOPE, preferably the DOPE is in (or adopts) the inverted hexagonal phase. DOPE can also be stimulated to form the inverted hexagonal phase in situ by a decrease in pH. In some aspects, this may be effected by including an appropriate pH-adjusting agent. Anionic lipids such as phosphatidic acid or cholesteryl hemisuccinate may also be used as fusogenic lipids, optionally in combination with neutral helper lipids such as DOPE, particularly if the therapeutic compound carries a positive charge. In this regard a cell penetrating peptide may also be used.
b) Cell penetrating peptides, which preferably have 2 to 12 amino acids, such as octaarginine, nonaarginine or octalysine, and/or are arginine rich cell penetrating peptides. These may advantageously be used in combination with other agents selected from the said one or more further compounds described herein, particularly in combination with fusogenic lipids such as DOPE.
c) Lysosomotropic agents, e.g. chloroquine.
d) Membrane-disruptive peptides, e.g. the peptides INF7, HSWYG, 43E (composed of three LAEL amino acid sequence units) and Histidine 10.
e) Membrane-disruptive polymers, e.g. polyethyleneimine.
f) Photochemical internalisation agents, e.g. a fluorescent label such as a fluorescein isothiocyanate, or a compound comprising it.
g) Agents that alter intracellular vesicle transport, e.g. by targeting factors which mediate the formation of transport vesicles (factors which mediate the formation of transport vesicles include Guanine nucleotide exchange factor— GBF1— which mediates the formation of transport vesicles by recruiting COPI coat proteins to cargo-bound receptor proteins found in the membrane of the Golgi; inhibition of GBF1 activity induces the retrograde movement of secretory proteins from the Golgi to the ER; the collapse of the Golgi into the ER triggers activation of unfolded protein response and ultimately causes apoptosis). An example of such an agent is Brefeldin A.

Said one or more further agents, if present, should be administered in combination with the bile acid/salt and the therapeutic compound—this can be separately, simultaneously or as part of a single composition. Preferably the bile acid/salt and therapeutic compound and said one or more further agents are all administered as part of the same composition. In one embodiment, said one or more further agents (or active part(s) thereof) may be chemically attached to the therapeutic compound (e.g. covalently, or alternatively conjugated non-covalently). This can help ensure that said one or more further agents (or active part(s) thereof) are duly incorporated into the cell with the therapeutic compound, i.e. where it is intended to have its effect. This approach is particularly preferred if photochemical internalisation is being used to promote efficacy.

As a further point, it is worth noting that although cell penetrating peptides are listed above as possible options for said one or more further agents, previously such peptide agents have also been described for use in enhancing the uptake of compounds into cells. Since the bile acid or salt performs that function in the present invention already, there is generally no need to include a cell penetrating peptide for this purpose alone, and in some aspects it is thus preferred for no such peptide agent to be present. Nonetheless, there may be instances where a cell penetrating peptide may advantageously be included in order to provide a useful effect once the therapeutic compound has been taken up into the cells and/or to further enhance the uptake of the therapeutic compound into the cells.

Typically just a single one of the above mentioned one or more further agents is used, though in some aspects it may be preferred to include two, three, four or even more.

In terms of how the bile acid/salt and/or the therapeutic compound are administered in accordance with the present invention, they are (each) typically formulated for administration with a pharmaceutically acceptable carrier or diluent. The bile acid/salt and/or the therapeutic compound may be administered in a variety of dosage forms. Thus, they may be administered orally, for example as tablets, troches, lozenges, aqueous or oily suspensions, dispersible powders or granules. They may also be administered parenterally, whether subcutaneously, intravenously, intramuscularly, intrasternally, transdermally or by infusion techniques. They may also be administered as suppositories. They may also be administered buccally, sublingually, rectally, topically, orally, nasally or via the pulmonary route, as either an aerosol spray or as drops.

By way of example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents, e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; mucolytic agents; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

The composition may be a liquid. Possible liquid compositions include solutions, suspensions and dispersions. Liquid formulations for oral administration may be syrups, emulsions and suspensions. The syrups may contain as carriers, for example, saccharose or saccharose with glycerine and/or mannitol and/or sorbitol.

Suspensions and emulsions may contain as carrier, for example, a natural gum, agar, sodium alginate, pectin, methylcellulose, carboxymethylcellulose, or polyvinyl alcohol. Suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water, or preferably they may be in the form of sterile, aqueous, isotonic saline solutions.

In a preferred embodiment of the invention, the bile acid/salt and the therapeutic compound are provided as a composition for the introduction of the therapeutic compound (preferably a macromolecule) into cells, wherein the therapeutic compound is mixed in solution with a bile salt, preferably a chenodeoxycholate salt, such as sodium chenodeoxycholate, and, optionally, said bile salt is employed in conjunction with EDTA, to this effect.

In another preferred embodiment of the invention the bile acid/salt, EDTA and the therapeutic compound are provided as a solid dry powder containing a mixture of the components, which after administration will form a solution in bodily fluid. Thus, in this embodiment, the invention provides a (dry) solid composition for the introduction of a therapeutic compound (preferably a macromolecule) into cells, comprising the therapeutic compound and a bile salt (preferably a chenodeoxycholate salt, such as sodium chenodeoxycholate), formulated as a capsule or as pellets, and, optionally, where said bile salt is employed in conjunction with EDTA, to this effect. The dry solid may be prepared e.g. by mixing the individual components together as dry powders, in appropriate proportions, or by first co-dissolving all of the components of the formulation in a liquid medium, and then drying the solution by any known method such as evaporation, vacuum drying or lyophilisation.

In another preferred embodiment, the invention provides a composition for the introduction of a therapeutic compound (typically a macromolecule) into cells, wherein the therapeutic compound and the bile acid/salt are combined in the form of a paste, ointment or gel, for example for topical application, and, optionally, wherein said bile salt is employed in conjunction with EDTA, to this effect.

Additional excipients can be included in the above compositions, such as one or more antioxidants, preservatives, dissolution aids, pH adjusters, and/or (in the case of oral or nasal administration) taste maskers.

Preferred agents for use in the compositions of the invention when the therapeutic compound and the bile acid/salt are present in solid form, are (i) fumed silica (aerosol) which may be used in amounts of e.g. 0.01 to 100 mg, preferably 0.1 to 10 mg, typically 0.5 to 5 mg; (ii) sodium starch glycholate which may be used in amounts of e.g. 0.1 to 500 mg, preferably 1 to 100 mg, typically 5 to 50 mg; (iii) one or more glidants, and/or (iv) one or more disintegrants.

Preferably, the bile acid or salt thereof is used to enable or enhance intracellular uptake of the therapeutic compound. Optionally, said bile salt is employed in conjunction with EDTA.

The present invention also provides a method of treatment of the human or animal body, which method comprises the step of: administering a therapeutic compound to the human or animal body together with together with a bile acid, wherein said bile acid is chenodeoxycholic acid or deoxycholic acid, wherein the bile acid or a pharmaceutically acceptable salt thereof is present in an amount that is sufficient to enable or enhance intracellular uptake of the therapeutic compound. Optionally, said bile salt is employed in conjunction with EDTA.

The present invention also provides for the use of a therapeutic compound and a bile acid or salt thereof in the manufacture of a medicament for use in a method of treatment of a human or animal body in need of the therapeutic compound. Preferably, the bile acid or salt thereof is present in an amount to enable or enhance intracellular uptake of the therapeutic compound. Optionally, said bile salt is employed in conjunction with EDTA

The present invention also provides a pharmaceutical or therapeutic composition comprising (a) a bile acid or a pharmaceutically acceptable salt thereof, wherein said bile acid is chenodeoxycholic acid or deoxycholic acid; and (b) a therapeutic compound. Preferably the composition also includes one or more further compounds selected from fusogenic lipids, cell penetrating peptides, lysosomotropic agents, membrane-disruptive peptides, membrane-disruptive polymers, photochemical internalisation agents, and agents that alter intracellular vesicle transport, and, optionally, said bile salt is employed in conjunction with EDTA, to this effect.

Desirably, in any formulation of the invention the bile acid or salt and therapeutic compound are encapsulated by a coating which (a) restricts dissolution of the bile acid or salt and therapeutic compound under aqueous conditions at a pH of 7.4, but ceases to restrict dissolution of the bile acid or salt and therapeutic compound at a pH which is lower than 7.4, and/or (b) contains one or more moieties capable of binding to a target cell population within the body.

In a first further preferred embodiment, the present invention provides a bile acid or a pharmaceutically acceptable salt thereof, for use in a method of treatment of the human or animal body, which method comprises administering a therapeutic compound to the human or animal body, wherein:
a) said bile acid or a pharmaceutically acceptable salt thereof is a salt of chenodeoxycholate, preferably sodium chenodeoxycholate,
b) said bile acid/salt and therapeutic compound are comprised in the same pharmaceutical composition
c) said composition is encapsulated by a coating which (a) restricts dissolution of the bile acid or salt and therapeutic compound under aqueous conditions at a pH of 7.4, but ceases to restrict dissolution of the bile acid or salt and therapeutic compound at a pH which is lower than 7.4, and/or (b) contains one or more moieties capable of binding to a target cell population within the body
d) said composition preferably being in liquid form, more preferably a form suitable for intravenous administration.

In a particularly preferred aspect of the above first further preferred embodiment, the bile acid/salt is formulated in the composition in combination with DOPE and PEG, such that the DOPE and/or PEG break down to release the therapeutic compound when exposed to a pH of less than 7.4, preferably less than 7.0, more preferably less than 6.5.

In a second further preferred embodiment, the present invention provides a bile acid or a pharmaceutically acceptable salt thereof, for use in a method of treatment of the human or animal body, which method comprises administering a therapeutic compound to the human or animal body, wherein:
a) said bile acid or a pharmaceutically acceptable salt thereof is a salt of chenodeoxycholate, preferably sodium chenodeoxycholate, and, optionally, said bile salt is employed in conjunction with EDTA,
b) in said method the bile acid or salt thereof is used to enable or enhance intracellular uptake of the therapeutic compound in the gut (wherein preferably bile acid/salt receptor-mediated internalisation occurs via clathrin-coated pits), and typically in the small intestine,
c) said therapeutic compound is a protein, for example a protein comprising a hydrophobic moiety (which is able to conjugate non-covalently to the chenodeoxycholate salt when in micellar form), and
d) wherein said method comprises the oral administration of a pharmaceutical composition comprising the bile salt, the protein and propyl gallate.

Suitable proteins for use in this embodiment include those listed above and/or used in the Examples.

Also for this second further preferred embodiment, it is preferred that the composition is formulated in a device (preferably an enteric-coated formulation, such as an enteric capsule) which resists dissolution at the pH found in the stomach but disintegrates at the pH found in the small intestine. For instance, the composition could be formulated within an enteric capsule which becomes permeable at a pH within the range of 5 to 6.

In a third further preferred embodiment, the present invention provides a bile acid or a pharmaceutically acceptable salt thereof, for use in a method of treatment of the human or animal body, which method comprises administering a therapeutic compound to the human or animal body, wherein:
a) said bile acid or a pharmaceutically acceptable salt thereof is a salt of chenodeoxycholate, preferably sodium chenodeoxycholate, and, optionally, said bile salt is employed in conjunction with EDTA,
b) said bile acid/salt and therapeutic compound are comprised in the same pharmaceutical composition,
c) said pharmaceutical composition is suitable for topical administration, and
d) said method involves the application of the composition to the skin.

Generally, the present invention is concerned with the treatment of humans, although in one aspect the invention concerns the treatment of non-human animals. Accordingly the subject in whom the present invention may find utility includes, by way of illustration: humans, mammals, companion animals and birds (with humans being the most preferred).

The following Examples illustrate the invention. They do not however limit the invention in any way.

The following Examples serve to illustrate the present invention, and should not be construed as limiting.

### EXAMPLES

### Example 1

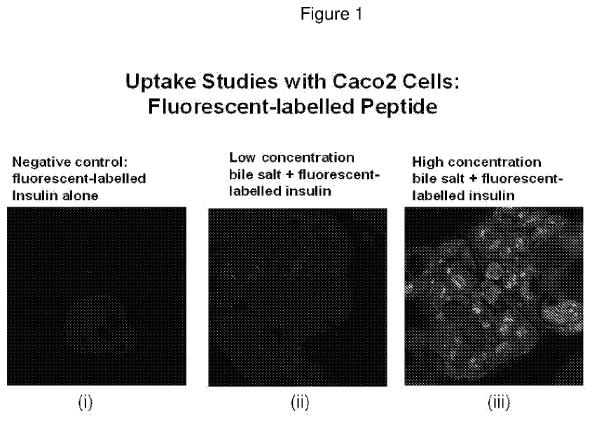

Caco-2 cells (passage #51) were cultured in DMEM (supplemented with 10% FBS) for three days on plastic coverslips at the bottom of 1 ml wells in 24-well cluster plates at a density of 1×106 cells/ml. 0.5 ml of medium was removed from each well, and replaced with medium containing (i) FITC-insulin alone (concentration 100 ug/ml), (ii) FITC-insulin with sodium chenodeoxycholate (1 mg/ml) or (iii) FITC-insulin with sodium chenodeoxycholate (2 mg/ml). The cells were incubated at 37° C. in 5% CO2 for half an hour. The supernatant was then removed, replaced with 0.5 ml of 4% paraformaldehyde solution, and incubated at room temperature for 15 minutes. The paraformaldehyde was then removed, the wells were washed three times with phosphate-buffered saline, and the coverslips then treated with mounting medium and viewed under a confocal microscope. Images are set out in FIG. 1. As can be seen from the photomicrographs, very little uptake was seen in cells treated with insulin alone, while uptake is enhanced by the presence of chenodeoxycholate in a concentration-dependent manner, fluorescent material clearly being seen in vacuoles within the cells at the higher concentration of the bile salt.

### Example 2

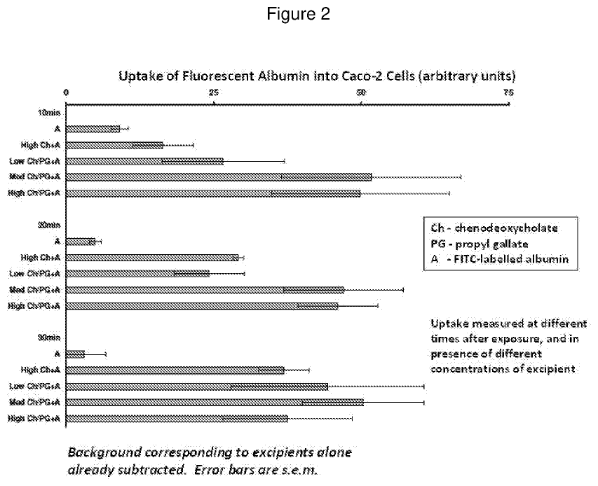

Caco-2 cells (passage #51) were cultured in DMEM (supplemented with 10% FBS) for four days at the bottom of 0.2 ml wells in 96-well cluster plates at a density of 2×105 cells/ml, with feeding where necessary. 0.2 ml of medium was removed from each well, and replaced with medium containing (i) FITC-albumin (bovine) alone (concentration 100 ug/ml) in row A, (ii) FITC-albumin 100 ug/ml with sodium chenodeoxycholate (High concentration: 1.33 mg/ml) in row B, (iii) FITC-albumin 100 ug/ml with sodium chenodeoxycholate/propyl gallate solution (0.66 and 0.33 mg/ml resp.— total solid 1 mg/ml) in row C, (iv) FITC-albumin 100 ug/ml with sodium chenodeoxycholate/propyl gallate solution (1.0 and 0.5 mg/ml resp.— total solid 1.5 mg/ml) in row D and (v) FITC-albumin 100 ug/ml with sodium chenodeoxycholate/propyl gallate solution (1.33 and 0.66 mg/ml resp.— total solid 2 mg/ml) in row E. The cells were incubated at 37° C. in 5% CO2 for ten minutes, and the supernatant was then removed from columns 1, 2 and 3 and washed gently 3 times with phosphate-buffered saline. The plate was read quickly in a Spectramax fluorescent plate-reader (excitation wavelength 494 nm, cutoff 515 nm, emission 515 nm). The plate was then incubated for a further ten minutes at 37° C. in 5% CO2, and the supernatant from wells 4, 5 and 6 removed with washing in PBS, before reading in the plate-reader as before. The plate was incubated for a further ten minutes, and then the supernatant from wells 7, 8 and 9 was removed and the wells washed and read as before. Readings indicative of the quantity of fluorescent material taken up by the cells for each group at each time point are shown in FIG. 2, where ‘Low’, ‘Med’ and ‘High’ refer to concentrations of chenodeoxycholate of 0.66, 1.0 and 0.33 mg/ml respectively. The results are shown in FIG. 2. As can be seen, the presence of chenodeoxycholate alone enhances the uptake by cells of fluorescent albumin, although when propyl gallate is included with the bile salt, the rate of uptake is more rapid, and reaches a maximal value much sooner (10 minutes, as compared to 30 minutes for chenodeoxycholate alone).

### Example 3

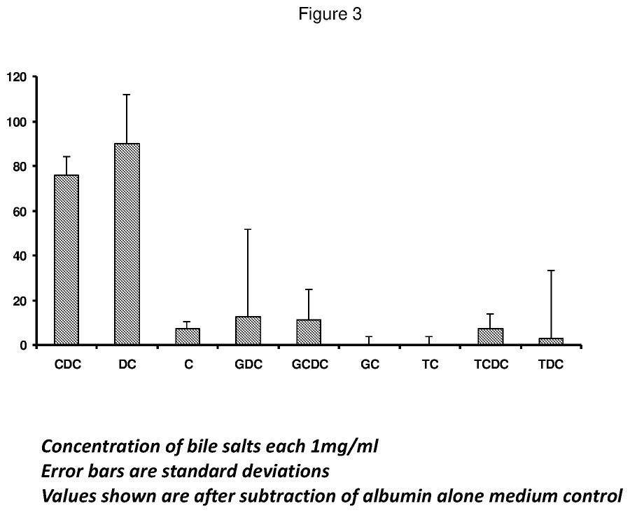

Caco-2 cells (passage #52) were cultured in DMEM (supplemented with 10% FBS) for four days at the bottom of 0.2 ml wells in 96-well cluster plates at a density of 2×105 cells/ml, with feeding where necessary. 0.2 ml of medium was removed from each well, and replaced with medium containing FITC-albumin (bovine) alone (concentration 100 ug/ml) or FITC-albumin 100 ug/ml with various bile salts at a concentration of 2 ug/ml. the cells were incubated at 37° C. in 5% CO2 for thirty minutes, and the supernatant was then removed from the wells, washed gently three times with phosphate-buffered saline (PBS) and then filled with 0.2 ml of PBS. The plate was read in a Spectramax fluorescent plate-reader (excitation wavelength 494 nm, cutoff 515 nm, emission 515 nm). The bile salts tested, all in the form of their sodium salts, were chenodeoxycholate, deoxycholate, cholate, glycodeoxycholate, glycochenodeoxycholate, glucocholate, taurocholate, taurochenodeoxycholate and taurodeoxycholate. The fluorescence readings obtained after subtraction of background consisting of cells incubated in medium alone, are shown in FIG. 3. Visual inspection of cells under the microscope in this and repeat experiments confirmed that, where fluorescence above background was observed, this was localised inside cells, often in discrete vacuoles. Uptake into cells is clearly evident after incubation with chenodeoxycholate and deoxycholate, while neither cholate nor taurocholate induce uptake. Subsequent experiments have demonstrated that, at concentrations both below and above the toxic limit, no uptake of labelled protein is seen with any natural conjugated bile salts (ie tauro- or glyco- derivatives).

### Example 4

Caco-2 cells (passage #56) were cultured in DMEM (supplemented with 10% FBS) for four days at the bottom of 0.2 ml wells in 96-well cluster plates at a density of 0.5×105 cells/ml, with feeding where necessary. 0.2 ml of medium was removed from each well, and replaced with 50 ul of medium lacking FBS, and containing chlorpromazine at different concentrations. Incubation was continued for ten minutes, then 50 ul of medium (without FBS) containing FITC-albumin (bovine) alone (concentration 200 ug/ml) or FITC-albumin 200 ug/ml with chenodeoxycholate was added, at varying concentrations. The cells were incubated at 37° C. in 5% CO2 for a further twenty minutes, and the supernatant was then removed from the wells, washed gently three times with medium without FBS, and then filled with 0.2 ml of medium (again without FBS). Uptake, or otherwise, of FITC-albumin into cells was assessed by visual inspection under a fluorescent microscope and scored according to fluorescence intensity. As can be seen in the table below setting out fluorescence scores based on the microscope images, concentration-dependent inhibition of uptake was observed upon preincubation with chlorpromazine, an inhibitor of formation of clathrin-coated pits. This contrasts with the results of similar experiments, preincubating with either amiloride (inhibitor of macropinocytosis) or nystatin (caveolin inhibitor), where no reduction in uptake was seen.

| Concentration | Concentration of Chlorpromazine | | | |
|---|---|---|---|---|
| of Cheno | (mg/ml) | | | |
| (mg/ml) | 0 | 2.5 | 5 | 10 |
| 1.0 | +++ | + | ++ | ± |
| 1.5 | ++ | ++ | ++ | − |

### Key

+++ high level of fluorescence
++ medium level of fluorescence
+ low level of fluorescence
□ very low/sporadic level of fluorescence
− no fluorescence

### Example 5

Caco-2 cells (passage #57) were cultured in DMEM (supplemented with 10% FBS) for four days at the bottom of 0.2 ml wells in 96-well cluster plates at a density of 0.5×105 cells/ml, with feeding where necessary. 0.2 ml of medium was removed from each well, and replaced with 50 ul of medium (lacking FBS) containing sodium ursodeoxycholate at different concentrations. Incubation was continued for ten minutes, then 50 ul of medium (-FBS) containing FITC-albumin (bovine) alone (concentration 100 ug/ml) or FITC-albumin 100 ug/ml with chenodeoxycholate was added, at varying concentrations. The cells were incubated at 37° C. in 5% CO2 for a further twenty minutes, and the supernatant was then removed from the wells, washed gently three times with medium without FBS and then filled with 0.2 ml of medium (minus FBS). Uptake, or otherwise, of FITC-albumin into cells was assessed by visual inspection under a fluorescent microscope and scored according to fluorescence intensity using the same grading scheme as in Example 4. As can be seen in the table below, concentration-dependent inhibition of uptake was observed upon preincubation with ursodeoxycholate.

| | Concentration of Chenodeoxycholate | | |
|---|---|---|---|
| | (mg/ml) | | |
| Samples tested | 1.5 | 1.0 | 0.5 |
| Cheno alone | +++ | +++ | + |
| Urso | ++ | ++ | − |
| preincubation | | | |
| 0.1 mg/ml | | | |
| Urso | ++ | + | − |
| preincubation | | | |
| 0.5 mg/ml | | | |
| Urso alone | − | − | − |

### Example 6

Caco-2 cells (passage #s 59 & 60) were cultured in DMEM (supplemented with 10% FBS) for four days at the bottom of 0.2 ml wells in 96-well cluster plates at a density of 0.5×105 cells/ml, with feeding where necessary. 0.2 ml of medium was removed from each well and replaced with 50 ul of medium (without FBS) containing either FITC-albumin (bovine) or FITC-Casein (concentration 100 ug/ml) alone, to which chenodeoxycholate was added, at varying concentrations. The cells were incubated at 37° C. in 5% CO2 for a further twenty minutes, and the supernatant was then removed from the wells, washed gently three times with medium without FBS, and then filled with 0.2 ml of medium (again without FBS). Uptake, or otherwise, of FITC-albumin into cells was assessed by visual inspection under a fluorescent microscope, and scored according to fluorescence intensity using the same grading scheme as in Example 4. The experiment was repeated on two separate occasions on different days. As can be seen in the table below, uptake of a similar extent was observed for both BSA and Casein, showing that uptake is not specific to a particular protein.

| | Concentration of | | | |
|---|---|---|---|---|
| | Chenodeoxycholate (mg/ml) | | | |
| | | | | Medium |
| Samples tested | 1.5 | 1.0 | 0.5 | Control |
| Chenowith FITC-BSA | | +++ | + | − |
| Cheno with FITC-Casein | | ++ | ± | − |
| Control | | − | − | − |
| Chenowith FITC-BSA | +++ | ++ | ± | − |
| Chenowith FITC-Casein | +++ | ++ | ± | − |
| Control | − | − | − | − |

### Example 7

Caco-2 cells (passage #60) were cultured in DMEM (supplemented with 10% FBS) for four days at the bottom of 0.2 ml wells in 96-well cluster plates at a density of 0.5×105 cells/ml, with feeding where necessary. 0.2 ml of medium was removed from each well, and replaced with 50 ul of medium (without FBS) containing FITC-albumin (bovine) alone (concentration 100 ug/ml) or FITC-albumin 200 ug/ml to which chenodeoxycholate alone, chenodeoxycholate:propyl gallate (2:1 wt:wt), taurocholate alone or taurocholate:propyl gallate (2:1 wt:wt) was added, at varying concentrations. The cells were incubated at 37° C. in 5% CO2 for a further twenty minutes, and the supernatant was then removed from the wells, washed gently three times with medium without FBS, and then filled with 0.2 ml of medium (again without FBS). Uptake, or otherwise, of FITC-albumin into cells was assessed by visual inspection under a fluorescent microscope and scored according to fluorescence intensity using the same grading scheme as in Example 4 (nd=not done/tested). As can be seen from the table below, the presence of propyl gallate appears to enhance uptake mediated by chenodeoxycholate, since at a concentration of bile salt of 1.0 mg/ml a higher level of uptake is seen with the cheno/PG combination than with cheno alone. In contrast, no uptake is seen for taurocholate, either in the presence or absence of PG. The effect of PG will be to encourage and strengthen the formation of bile salt micelles. Chenodeoxycholate has a low CMC, and micelles will form at the low concentrations tested here. However, strengthening the micelle increases the uptake of protein as expected, in accord with the mechanism of uptake, in which the receptor-mediated process recognises bile salts in the micellar form. However, bile salt micelle formation alone is not sufficient condition for this uptake to occur, since even in the presence of PG, no uptake is observed in the presence of taurocholate.

| | Concentration of Bile Salt mg/ml) | | | | | |
|---|---|---|---|---|---|---|
| | | | | | | Medium |
| Samples tested | 1.5 | 1.0 | 0.66 | 0.5 | 0.33 | Control |
| Cheno alone | +++ | ++ | nd | ± | nd | − |
| Cheno/PG | | +++ | ++ | nd | − | − |
| Taurocholate | − | − | − | − | − | − |
| alone | | | | | | |
| Taurocholate/PG | − | − | − | − | − | − |

### Example 8

Caco-2 cells (passage #s 60) were cultured in DMEM (supplemented with 10% FBS) for four days at the bottom of 0.2 ml wells in 96-well cluster plates at a density of 0.5×105 cells/ml, with feeding where necessary. 0.2 ml of medium was removed from each well and replaced with 50 ul of medium (without FBS) containing FITC-albumin (bovine), to which chenodeoxycholate was added, at a concentration of 0.5 mg/ml, either alone, or in combination with sodium EDTA, or orthophenanthroline, at a concentration of 5 mg/ml. The cells were incubated at 37° C. in 5% CO2 for a further twenty-five minutes, and the supernatant was then removed from the wells, washed gently three times with medium without FBS, and then filled with 0.2 ml of medium (again without FBS). Uptake, or otherwise, of FITC-albumin into cells was assessed by visual inspection under a fluorescent microscope and scored according to fluorescence intensity using the same grading scheme as in Example 4. It can be seen from the table below that at the concentrations employed, neither chenodeoxycholate nor EDTA were able to enhance uptake of the protein on their own. However, in combination, significant uptake was observed, showing that EDTA is able to synergise with chenodeoxycholate in stimulating uptake of proteins by intestinal cells. Another chelating agent, however, o-phenanthroline, does not have this effect.

| | Concentration of | |
|---|---|---|
| | Chenodeoxycholate | |
| Samples tested | 0.5 mg/ml | 0 mg/ml |
| Cheno alone | + | ± |
| Cheno/EDTA | +++ | ± |
| Cheno/o-phenanthroline | − | − |

### Example 9

The experiment was repeated as described in Example 8, except that the concentrations of chenodeoxycholate were 0, 0.5 and 1 mg/ml, and that EDTA, DTPA and EGTA were employed at a concentration of 1 mg/ml. Incubation time was 30 minutes. As can be seen in the table below, enhancement of uptake over and above that seen with chenodeoxycholate alone was seen when EDTA is added, but not with DTPA or EGTA. Similar observations were made when Cheno was added first, incubated for 20 minutes, then replaced by EDTA for ten minutes.

| | Concentration of | | |
|---|---|---|---|
| | Chenodeoxycholate | | |
| Samples tested | 1 mg/ml | 0.5 mg/ml | 0 mg/ml |
| Cheno alone | ++ | + | − |
| Cheno/EDTA | +++ | ++ | − |
| Cheno/DTPA | + | + | − |
| Cheno/EGTA | + | + | − |

### Example 10

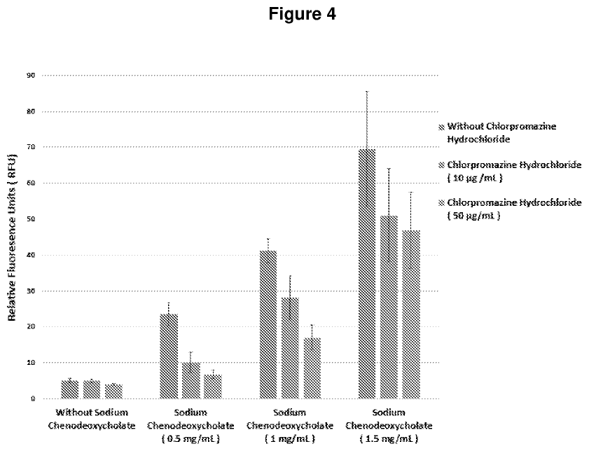

Caco-2 cells (passage 61) were cultured in DMEM (supplemented with 10% FBS) for four days at the bottom of 0.2 ml wells in 96-well black cluster plates at a density of 0.5×10⁵ cells/ml, with feeding where necessary. 0.2 ml of medium was removed from each well and replaced with 50 ul of medium (without FBS) containing chlorpromazine at different concentrations. After 20 minutes, 100 ul of FITC-albumin (bovine) alone (concentration 100 ug/ml) or FITC-albumin containing chenodeoxycholate at different concentrations was added. The cells were incubated at 37° C. in 5% CO2 for a further twenty minutes, and the supernatant was then removed from the wells, washed gently three times with medium without FBS, and then filled with 0.2 ml of medium (again without FBS). Uptake, or otherwise, of FITC-albumin into cells was measured in a TECAM plate reader (excitation wavelength 492 nm, emission wavelength 525 nm). Results for uptake of FITC-BSA shown in the chart in FIG. 4 demonstrate that chlorpromazine (at both high and low concentrations) inhibits uptake stimulated by the bile salt at all concentrations. This confirms the results of the experiment described in Example 4, where concentration-dependent inhibition of uptake was observed upon preincubation with chlorpromazine, an inhibitor of formation of clathrin-coated pits.

### Example 11

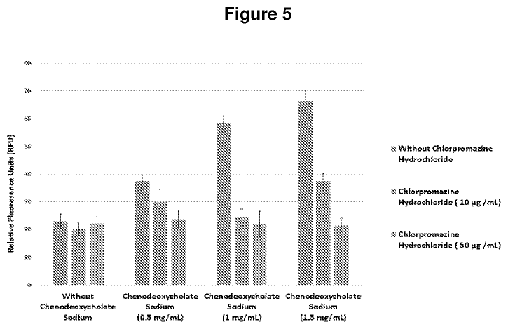

Caco-2 cells (passage 61) were cultured in DMEM (supplemented with 10% FBS) for four days in culture flasks at a density of 0.5×10⁵ cells/ml, with feeding where necessary. Cells were then trypsinised and suspended and washed by centrifugation in medium free of FBS to give a suspension with a final concentration of cells of 1.3×10⁶ cells per ml. 0.4 ml suspension was dispensed into each of eleven 1.5 ml plastic Eppendorf vials, and 40 ul of medium (without FBS) containing chlorpromazine at different concentrations was added to each vial, followed by incubation at 37° C. for 20 minutes. 400 ul of FITC-albumin (bovine) alone (concentration 100 ug/ml) or FITC-albumin containing chenodeoxycholate at different concentrations was then added. The suspensions were incubated at 37° C. for a further twenty minutes, and the cells then washed gently by centrifugation three times with medium without FBS. The pellet was then resuspended in 600 ul of medium (again without FBS) and 200 ul transferred to each of three wells of a black 98-well microplate. Uptake, or otherwise, of FITC-albumin into cells was measured in a Spectramax Gemini fluorescent plate reader plate reader (excitation wavelength 492 nm, emission wavelength 525 nm). Results for uptake of FITC-BSA shown in the chart in FIG. 5 demonstrate that chlorpromazine (at both high and low concentrations) inhibits uptake stimulated by the bile salt at all concentrations. This confirms the findings shown in experiment 10 and demonstrates that measurement of uptake by cells in suspension gives very similar results to experiments conducted with cells adherent to a plastic surface.

### Example 12

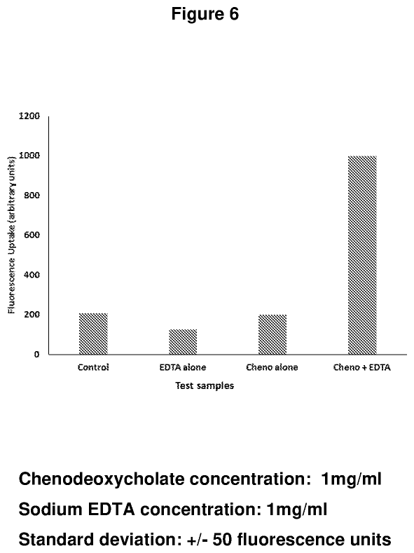

Caco-2 cells (passage 61) were cultured in DMEM (supplemented with 10% FBS) for four days in culture flasks at a density of 0.5×10⁵ cells/ml, with feeding where necessary. Cells were then trypsinised and suspended and washed by centrifugation in medium free of FBS to give a suspension with a final concentration of cells of 1×10⁶ cells per ml. 1 ml suspension was dispensed into 1.5 ml plastic Eppendorf vials, and the cells centrifuged in medium (without FBS) and resuspended in 100 ul volume. 1 ml of FITC-albumin (bovine) alone (concentration 100 ug/ml) or FITC-albumin containing chenodeoxycholate and EDTA alone or in combination at concentrations of 1 mg/ml were then added. The suspensions were incubated at 37° C. for a further 15 minutes, and the cells then washed gently by centrifugation three times with medium without FBS. The pellet was then resuspended in 600 ul of medium (again without FBS) and 200 ul transferred to each of three wells of a black 98-well microplate. Uptake, or otherwise, of FITC-albumin into cells was measured in a Spectramax Gemini fluorescent plate reader plate reader (excitation wavelength 492 nm, emission wavelength 525 nm). In this experiment, the concentrations of the two agents (chenodeoxycholate and EDTA) individually were too low to enhance uptake of the protein within the timeframe tested. However, the results shown in FIG. 6 show that when the two agents are combined, a very significant enhancement of uptake occurs.

### Example 13

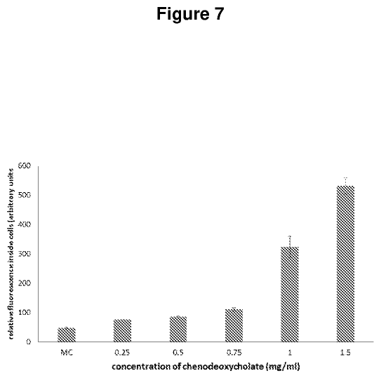

Caco-2 cells (passage 61) were cultured in DMEM (supplemented with 10% FBS) for four days in culture flasks at a density of 0.5×10⁵ cells/ml, with feeding where necessary. Cells were then trypsinised and suspended and washed by centrifugation in medium free of FBS to give a suspension with a final concentration of cells of 1×10⁶ cells per ml. 1 ml suspension was dispensed into 1.5 ml plastic Eppendorf vials, and the cells centrifuged in medium (without FBS) and resuspended in 100 ul volume. 1 ml of FITC-hGH alone (concentration 100 ug/ml) or FITC-hGH containing chenodeoxycholate at different concentrations were then added. The suspensions were incubated at 37° C. for a further 15 minutes, and the cells then washed gently by centrifugation three times with medium without FBS. The pellet was then resuspended in 600 ul of medium (again without FBS) and 200 ul transferred to each of three wells of a black 98-well microplate. Uptake, or otherwise, of FITC-hGH into cells was measured in a Spectramax Gemini fluorescent plate reader plate reader (excitation wavelength 492 nm, emission wavelength 525 nm). The data shown in FIG. 7 demonstrates that chenodeoxycholate enhances the uptake of hGH into cells in a dose-dependent manner similar to that seen for uptake of other proteins such as insulin, BSA and casein.

### Example 14

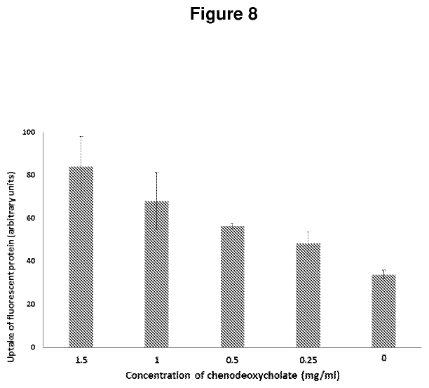

Cells of the human intestinal IEC6 cell line (passage 48) were cultured in DMEM (supplemented with 10% FBS) for four days in culture flasks at a density of 0.5×10⁵ cells/ml, with feeding where necessary. Cells were then trypsinised and suspended and washed by centrifugation in medium free of FBS to give a suspension with a final concentration of cells of 1×10⁶ cells per ml. 1 ml suspension was dispensed into 1.5 ml plastic Eppendorf vials, and the cells centrifuged in medium (without FBS) and resuspended in 100 ul volume. 1 ml of FITC-albumin (bovine) alone (concentration 100 ug/ml) or FITC-albumin containing chenodeoxycholate alone or at different concentrations were then added. The suspensions were incubated at 37° C. for a further 15 minutes, and the cells then washed gently by centrifugation three times with medium without FBS. The pellet was then resuspended in 600 ul of medium (again without FBS) and 200 ul transferred to each of three wells of a black 98-well microplate. Uptake, or otherwise, of FITC-albumin into cells was measured in a Spectramax Gemini fluorescent plate reader plate reader (excitation wavelength 492 nm, emission wavelength 525 nm). The results shown in FIG. 8 demonstrate that, in a similar manner to that seen for Caco-2 cells, uptake of protein into IEC6 cells is enhanced by the presence of chenodeoxycholate, confirming that this is a phenomenon common to intestinal cells in general.

### Example 15

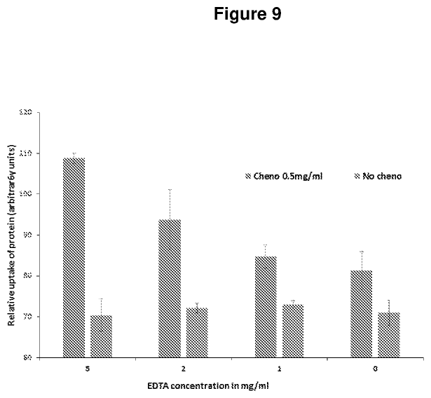

Cells of the human intestinal IEC6 cell line (passage 48) were cultured in DMEM (supplemented with 10% FBS) for four days in culture flasks at a density of 0.5×10⁵ cells/ml, with feeding where necessary. Cells were then trypsinised and suspended and washed by centrifugation in medium free of FBS to give a suspension with a final concentration of cells of 1×10⁶ cells per ml. 1 ml suspension was dispensed into 1.5 ml plastic Eppendorf vials, and the cells centrifuged in medium (without FBS) and resuspended in 100 ul volume. 1 ml of FITC-albumin (bovine) alone (concentration 100 ug/ml) or FITC-albumin containing chenodeoxycholate at a concentration of 0.5 mg/ml, and different concentrations of EDTA were then added. The suspensions were incubated at 37° C. for a further 15 minutes, and the cells then washed gently by centrifugation three times with medium without FBS. The pellet was then resuspended in 600 ul of medium (again without FBS) and 200 ul transferred to each of three wells of a black 98-well microplate. Uptake, or otherwise, of FITC-albumin into cells was measured in a Spectramax Gemini fluorescent plate reader plate reader (excitation wavelength 492 nm, emission wavelength 525 nm). The results shown in FIG. 9 demonstrate that, in IEC6 cells enhancement of uptake of BSA by chenodeoxycholate is augmented by EDTA in a dose-dependent fashion.

1. A bile acid or a pharmaceutically acceptable salt thereof, for use in a method of treatment of the human or animal body, which method comprises administering a therapeutic compound to the human or animal body, wherein:
a) said bile acid is chenodeoxycholic acid or deoxycholic acid, and
b) said method the bile acid or salt is used to enable or enhance intracellular uptake of the therapeutic compound.

2. A bile acid or salt according to claim 1, for use in a method as defined in claim 1, wherein said bile salt is employed in conjunction with EDTA.

3. A bile acid or salt according to claim 1, for use in a method as defined in claim 1, wherein said therapeutic compound is a macromolecule or a protein.

4. (canceled)

5. A bile acid or salt according to any one of claim 1, for use in a method as defined in any one of claim 1, wherein said bile acid or pharmaceutically acceptable salt thereof is a chenodeoxycholate salt, preferably sodium chenodeoxycholate.

6. A bile acid or salt according to claim 1, for use in the method as defined in claim 1, wherein in said method, the concentration of the bile acid or salt which is achieved at the surface of the cells into which intracellular uptake is to be enabled or enhanced, is from 2 mg/ml to 40 mg/ml.

7. A bile acid or salt according to claim 2, for use in a method as defined in claim 2, wherein in said method, the concentration of the EDTA which is achieved at the surface of the cells into which intracellular uptake is to be enabled or enhanced, is from 0.1 mg/ml to 10 mg/ml.

8. (canceled)

9. A bile acid or salt according to claim 1, for use in a method as defined in claim 1, wherein in said method bile acid or salt receptor-mediated internalisation via clathrin-coated pits is used to enable or enhance intracellular uptake of the therapeutic compound.

10. A bile acid or salt according to claim 1, for use in a method as defined in claim 1, wherein in said use of the bile acid or salt, said bile acid or salt is in micellar form.

11. A bile acid or salt according to claim 1, for use in a method as defined in claim 1, wherein in said method the bile acid or salt thereof is used in combination with one or more additional agents.

12. A bile acid or salt according to claim 11, for use in a method as defined in claim 11, wherein in said use of the bile acid or salt, said bile acid or salt is in micellar form, and said one or more additional agents comprise one or more agents which:
a) stabilize said micellar form of the bile acid or salt thereof; and/or
b) enable or enhance the formation of said micellar form.

13. A bile acid or salt according to claim 11, for use in a method as defined in claim 11, wherein said one or more agents include propyl gallate.

14. A bile acid or salt according to claim 11, for use in a method as defined in claim 11, wherein said one or more additional agents comprise one or more agents selected from:
a) a fusogenic lipid, which is preferably selected from cationic liposomes, neutral helper lipids such as DOPE, and anionic lipids such as phosphatidic acid;
b) a cell penetrating peptide, which is preferably a cell penetrating peptide having 2 to 12 amino acids, such as octaarginine, nonaarginine and octalysine;
c) a lysosomotropic agent, which is preferably chloroquine;
d) a membrane-disruptive peptide which is preferably INF7, H5WYG, 43E or Histidine 10;
e) a membrane-disruptive polymer, which is preferably polyethyleneimine;
f) a photochemical internalisation agent, which is preferably fluoroscein isothiocyanate or a compound comprising it; and/or
g) an agent that alters intracellular vesicle transport, which is preferably Brefeldin A.

15. A bile acid or salt according to claim 1, for use in a method as defined in claim 1, wherein the therapeutic compound is a nucleic acid polymer and the method of treatment is a method of gene therapy.

16. A bile acid or salt according to claim 1, for use in a method as defined in claim 1, wherein said therapeutic compound comprises a hydrophobic moiety.

17. A bile acid or salt according to claim 1, for use in a method as defined in claim 1, wherein in said method, the bile acid or salt thereof is used in the form of micelles conjugated non-covalently to the therapeutic compound, to enable or enhance intracellular uptake of the therapeutic compound.

18. A bile acid or salt according to claim 1, for use in a method as defined in claim 1, wherein in said method, the bile acid or salt thereof is used to enable or enhance intracellular uptake of one or more therapeutic compounds located in the vicinity of the cell.

19. A bile acid or salt according to claim 1, for use in a method as defined in claim 1, wherein said method comprises administering a composition comprising the bile acid or salt thereof and the therapeutic compound, and, if one or more additional agents are being used in combination with the bile acid or salt thereof in said method, preferably also one or more of said one or more additional agents.

20. A bile acid or salt according to claim 19, for use in a method as defined in claim 1, wherein said therapeutic compound comprises a hydrophobic moiety, and wherein the weight ratio of the bile acid or salt:the therapeutic compound in said composition is from 20:1 to 5:1.

21. A bile acid or salt according to claim 19, for use in a method as defined in claim 19, wherein said composition is in the form of a solution, suspension or dispersion.

22. A bile acid or salt according to claim 19, for use in a method as defined in claim 19, wherein said composition is in the form of a paste, ointment, gel, or powder, said powder being comprised in a capsule or pellet.

23. (canceled)

24. A bile acid or salt according to claim 21, for use in a method as defined in claim 21 wherein said composition is a composition for intravenous administration, and the bile acid or salt and therapeutic compound in the composition are encapsulated by a coating which restricts dissolution of the bile acid or salt and therapeutic compound, wherein the coating (a) ceases to restrict dissolution of the bile acid or salt and therapeutic compound at a pH of less than 7.4, and/or (b) contains one or more moieties capable of binding to a target cell population within the body.

25-28. (canceled)

29. A pharmaceutical composition comprising:
a) a bile acid or a pharmaceutically acceptable salt thereof, wherein said bile acid is chenodeoxycholic acid or deoxycholic acid;
b) a therapeutic compound; and
c) one or more further compounds selected from fusogenic lipids, cell penetrating peptides, lysosomotropic agents, membrane-disruptive peptides, membrane-disruptive polymers, photochemical internalisation agents, and agents that alter intracellular vesicle transport.

30. A pharmaceutical composition according to claim 29, wherein said bile salt is employed in conjunction with EDTA.

31. A pharmaceutical composition comprising:
a) a bile acid or a pharmaceutically acceptable salt thereof, wherein said bile acid is chenodeoxycholic acid or deoxycholic acid; and
b) a therapeutic compound;
c) wherein the bile acid or salt and therapeutic compound are encapsulated by a coating which (a) restricts dissolution of the bile acid or salt and therapeutic compound under aqueous conditions at a pH of 7.4 but ceases to restrict dissolution of the bile acid or salt and therapeutic compound at a pH which is lower than 7.4, and/or (b) contains one or more moieties capable of binding to a target cell population within the body.

32. A pharmaceutical composition according to claim 31, wherein said bile salt is employed in conjunction with EDTA.

33. A composition according to claim 32, wherein the composition further comprises one or more additional agents which:
a) stabilize said micellar form of the bile acid or salt thereof; and/or
b) enable or enhance the formation of said micellar form.

34. A composition according to claim 29, wherein said bile acid or pharmaceutically acceptable salt thereof is a chenodeoxycholate salt, preferably sodium chenodeoxycholate.

35. A composition according to claim 29, wherein said therapeutic compound is a macromolecule or a protein.

36. (canceled)

37. A composition according to claim 29, wherein the ratio of the bile acid or salt to the therapeutic compound is from 20:1 to 5:1.

38. A composition according to claim 29, wherein the concentration of the EDTA which is achieved at the surface of the cells into which intracellular uptake is to be enabled or enhanced, is from 0.1 mg/ml to 10 mg/ml.

39. (canceled)
